(19) United States
(12) Patent Application Publication
(10) Pub. No.: US 2023/0009969 A1
(43) Pub. Date: Jan. 12, 2023

(54) PREPARATION METHOD FOR SYNTHESIZING S-NICOTINE FROM GLUTARATE

(71) Applicant: SHENZHEN ZINWI BIO-TECH CO., LTD, Guangdong (CN)

(72) Inventors: Jun ZOU, Guangdong (CN); Yang ZOU, Guangdong (CN); Meisen LIU, Guangdong (CN); Weixian LUO, Guangdong (CN)

(73) Assignee: SHENZHEN ZINWI BIO-TECH CO., LTD, Guangdong (CN)

(21) Appl. No.: 17/547,247

(22) Filed: Dec. 10, 2021

Related U.S. Application Data
(63) Continuation of application No. PCT/CN2021/112805, filed on Aug. 16, 2021.

(30) Foreign Application Priority Data
Jul. 10, 2021 (CN) 202110781162.3

(51) Int. Cl. C07D 401/04

(52) U.S. Cl. CPC C07D 401/04

(57) ABSTRACT

The present invention provides a preparation method for synthesizing S-nicotine from glutarate, including: reacting nicotinate with glutarate in the presence of a base catalyst to obtain 5-carbonyl-5-(pyridin-3-yl)pentanoic acid, reacting with an amination reagent to obtain 5-oxo-5-(pyridin-3-yl)pentanamide, performing Hofmann degradation on to obtain 4-amino-1-(pyridin-3-yl)butanone, reducing a carbonyl group of the 4-amino-1-(pyridin-3-yl)butanone by using (+)-B-diisopinocampheyl chloroborane to obtain (S)-4-amino-1-(pyridin-3-yl)butan-1-ol, performing chlorination and cyclization to obtain S-demethylnicotine, and finally performing amine methylation to obtain S-nicotine.

### CROSS-REFERENCE TO RELATED APPLICATION

This application is a continuation of international application of PCT application serial no. PCT/CN2021/112805 filed on Aug. 16, 2021, which claims the priority benefit of China application no. 202110781162.3, filed on Jul. 10, 2021. The entirety of each of the above mentioned patent applications is hereby incorporated by reference herein and made a part of this specification.

### BACKGROUND

### Technical Field

The present invention relates to the technical field of chemical synthesis, and particularly relates to a preparation method for synthesizing S-nicotine from glutarate.

Nicotine, as one of the important active ingredients of e-cigarette, is mainly extracted from tobaccos or artificially synthesized by chemical methods. Nicotine extracted and purified from plants such as tobaccos also contains other carcinogenic tobacco compound impurities that are harmful to human health; and furthermore, tobacco extracts are affected by raw materials and climate, so that it is difficult to industrially produce on a large scale. Nicotine artificially synthesized by chemical methods is almost free of other carcinogenic tobacco compound impurities, and can be industrially produced on a large scale.

A method for preparing nicotine from 3-bromopyridine is reported in Journal of Heterocyclic Chemistry, 2009, 46(6), 1252-1258., as shown in Reaction Formula 1:

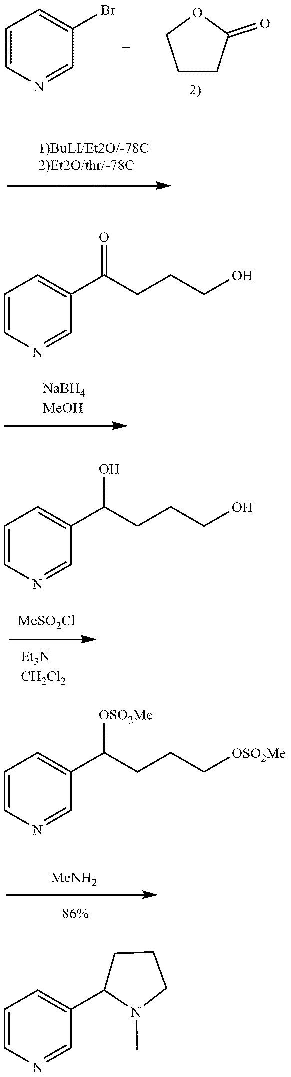

According to the preparation method of Reaction Formula 1, 3-bromopyridine is used as a starting material, which is expensive and requires an ultra-low temperature (-78° C.) condition, and the experimental conditions are harsh, so that the preparation method is not suitable for industrial production, and produced nicotine is racemic nicotine.

At present, there are few preparation methods of nicotine in a single configuration with optical activity. A patent with a publication No. CN104341390A discloses a preparation method of S-nicotine. According to the method, cyclic imine is used as a starting material, an expensive chiral catalyst is required, high-pressure hydrogen equipment is required, and the production cost is relatively high, so that the method is not suitable for large-scale industrial production. A patent with a publication No. CN111233829A discloses a preparation method of nicotine with optical activity. According to the method, a chiral ligand containing nitrogen or phosphorus is used to prepare an organometallic catalyst, an imide derivative is used as a starting material to prepare S-nicotine, the preparation of the organometallic catalyst is relatively complicated, the production cost is relatively high, and the purity of S-nicotine is relatively low.

Glutarate is a widely available and inexpensive raw material, but there is no report on the industrial synthesis of S-nicotine by using glutarate as a raw material.

### SUMMARY

In order to reduce the preparation cost of S-nicotine, the present application provides a preparation method for synthesizing S-nicotine from glutarate.

In a first aspect, the present application provides a preparation method for synthesizing S-nicotine from glutarate, which is implemented by adopting the following technical solutions.

A preparation method for synthesizing S-nicotine from glutarate, including the following steps:
Step S1: performing a condensation reaction on nicotinate and glutarate in the presence of a base catalyst to obtain 5-carbonyl-5-(pyridin-3-yl)pentanoic acid;
Step S2: reacting the 5-carbonyl-5-(pyridin-3-yl)pentanoic acid with an amination reagent to obtain 5-oxo-5-(pyridin-3-yl)pentanamide;
Step S3: performing a Hofmann degradation reaction on the 5-oxo-5-(pyridin-3-yl) pentanamide in the presence of hypochlorite to obtain 4-amino-1-(pyridin-3-yl)butanone;
Step S4: adding the 4-amino-1-(pyridin-3-yl)butanone and (+)-B-diisopinocampheyl chloroborane into an organic solvent II, and reacting at -30 to 10° C. to obtain (S)-4-amino-1-(pyridin-3-yl)butan-1-ol;
Step S5: reacting the (S)-4-amino-1-(pyridin-3-yl)butan-1-ol with a chlorination reagent to obtain (S)-4-amino-1-(pyridin-3-yl)chloro-butane;
Step S6: performing a cyclization reaction on (S)-4-amino-1-(pyridin-3-yl)butyl-1-chloride in the presence of a base to obtain S-demethylnicotine; and
Step S7. reacting the S-demethylnicotine with an amine methylation reagent to obtain S-nicotine.

By adopting the above technical solutions, nicotinate and glutarate are inexpensive raw materials with a wide source, so that the production cost of the raw materials can be reduced; a Claisen condensation reaction is performed on the nicotinate and the glutarate in the presence of a base catalyst to obtain 5-carbonyl-5-(pyridin-3-yl)pentanoic acid, a reaction with an amination reagent is performed to obtain 5-oxo-5-(pyridin-3-yl)pentanamide, Hofmann degradation is performed to obtain 4-amino-1-(pyridin-3-yl)butanone; a carbonyl group of the 4-amino-1-(pyridin-3-yl)butanone is reduced by using (+)-B-diisopinocampheyl chloroborane to induce the production of a chiral hydroxyl group so as to obtain (S)-4-amino-1-(pyridin-3-yl)butan-1-ol, chlorination and cyclization are performed to form chiral S-demethylnicotine, and finally amine methylation is performed to obtain S-nicotine with photochemical activity. A synthetic route using glutarate as a raw material of the present application has the advantages of simple operation, readily available raw materials, high yield, high ee value of S-nicotine, milder reaction conditions, simple treatment processes in the reaction process, etc., and is more suitable for industrial production.

Preferably, at Step S1, a molar ratio of the nicotinate to the glutarate to the base catalyst is 1: (1-1.5): (1.2-2); and more preferably, the molar ratio of the nicotinate to the glutarate to the base catalyst is 1: 1.5: 2.

In the present application, at Step S1, the glutarate is selected from any one of diethyl glutarate, dimethyl glutarate, di-n-propyl glutarate, and di-n-pentyl glutarate; and from the perspective of reaction cost, the cost of the glutarate being diethyl glutarate or dimethyl glutarate is lower.

In the present application, at Step S1, the nicotinate is methyl nicotinate or ethyl nicotinate.

In the present application, at Step S1, the reaction temperature of the glutarate and the base catalyst is 0 to 5° C. and is preferably 0° C., the reaction time is 30 min; and the reaction temperature after the nicotinate is added is 20 to 30° C. and is preferably 25° C.

In the present application, the solvent used at Step S1 may be one or more of tetrahydrofuran, methyl tertiary butyl ether, dimethyl tetrahydrofuran, and 1,4-dioxane; and preferably, the organic solvent I is tetrahydrofuran.

In the present application, at Step S1, the base catalyst is selected from one or more of alkali metal alkoxide, alkaline earth metal hydride, alkaline earth metal oxide, amine, a metal salt of amine, hydroxide, carbonate, and bicarbonate.

In the present application, the alkali metal alkoxide includes, but is not limited to, any one of sodium tert-butoxide, sodium methoxide, sodium ethoxide, and potassium tert-butoxide.

In the present application, the alkaline earth metal hydride includes, but is not limited to, one or more of NaH, LiH, and KH.

In the present application, the alkaline earth metal oxide includes, but is not limited to, one or more of Na₂O, Li₂O, and K₂O.

In the present application, the amine includes, but is not limited to, triethylamine and/or diisopropylethyl amine.

In the present application, the metal salt of amine includes, but is not limited to, sodium bis(trimethylsilyl)amide and/or lithium diisopropylamide.

In the present application, the hydroxide includes, but is not limited to, one or more of sodium hydroxide, lithium hydroxide, and magnesium hydroxide.

In the present application, the carbonate includes, but is not limited to, one or more of sodium carbonate, potassium carbonate, and cesium carbonate.

In the present application, the bicarbonate includes, but is not limited to, sodium bicarbonate and/or potassium bicarbonate.

More preferably, the base catalyst is selected from any one of sodium tert-butoxide, NaH, and potassium tert-butoxide.

In the present application, a mixture containing 5-carbonyl-5-(pyridin-3-yl)pentanoic acid is obtained at Step S1.

In the present application, at Step S2, the pH of the system needs to be adjusted before the 5-carbonyl-5-(pyridin-3-yl)pentanoic acid reacts with the amination reagent, specifically, the pH of the mixture containing 5-carbonyl-5-(pyridin-3-yl)pentanoic acid obtained at Step S1 is adjusted to 2 to 5, and preferably the pH of the system is adjust to 4 by using hydrochloric acid.

Preferably, at Step S2, the amination reagent is selected from one or more of ammonium hydroxide, formamide, and acetamide; when the amination reagent is ammonium hydroxide, the price is lower, the production cost of ammonium hydroxide is lower than the production cost of formamide and acetamide, and a subsequent deformylation and deacetylation step is not required, the reaction steps are less, which is more conducive to industrial production.

Preferably, at Step S2, a molar ratio of the 5-carbonyl-5-(pyridin-3-yl)pentanoic acid to the ammonium hydroxide is 1: (2-4); and more preferably, the molar ratio of the 5-carbonyl-5-(pyridin-3-yl)pentanoic acid to the ammonium hydroxide is 1: 3.

In the present application, the reaction temperature of Step S2 is 60 to 70° C., and the reaction time is 1 to 3 h; and preferably, the reaction temperature of Step S2 is 65° C., and the reaction time is 2 h.

In the present application, a mixture containing 5-oxo-5-(pyridin-3-yl)pentanamide is obtained at Step S2.

In the present application, at Step S3, the hypochlorite is selected from any one of sodium hypochlorite, sodium hypobromite, and potassium hypochlorite; and preferably, at Step S3, the hypochlorite is sodium hypochlorite.

In the present application, at Step S3, a molar ratio of the 5-oxo-5-(pyridin-3-yl)pentanamide to the hypochlorite is 1: (1-2); and preferably, the molar ratio of the 5-oxo-5-(pyridin-3-yl)pentanamide to the hypochlorite is 1: 1.5.

In the present application, at Step S3, specifically, the mixture containing 5-oxo-5-(pyridin-3-yl)pentanamide obtained at Step S2 is quickly added into the hypochlorite at 0° C., a reaction is performed at 0° C. for 1 h, the temperature is raised to 71° C., the reaction is continued at 71° C. for 1 h, after the reaction is stopped, the reaction solution is cooled to 25° C. and added with a saturated NaOH aqueous solution, extraction is performed, an organic phase is taken, dried, and subjected to rotary evaporation for removing the solvent to obtain the 4-amino-1-(pyridin-3-yl)butanone.

Preferably, at Step S4, a molar ratio of the 4-amino-1-(pyridin-3-yl)butanone to the (+)-B-diisopinocampheyl chloroborane is 1: (1.2-2); and more preferably, the molar ratio of the 4-amino-1-(pyridin-3-yl)butanone to the (+)-B-diisopinocampheyl chloroborane is 1: 1.5.

Preferably, at Step S4, the organic solvent is tetrahydrofuran.

Preferably, the reaction temperature of Step S4 is 0° C.

In the present application, the reaction time of Step S4 is 2 h.

In the present application, a mixture containing (S)-4-amino-1-(pyridin-3-yl)butan-1-ol is obtained at Step S4.

Preferably, at Step S5, the chlorination reagent is selected from one or more of oxalyl chloride, thionyl chloride, and trichlorophosphorus; and more preferably, the chlorination reagent is oxalyl chloride.

Preferably, at Step S5, a molar ratio of the (S)-4-amino-1-(pyridin-3-yl)butan-1-ol to the oxalyl chloride is 1: (1-1.5); and more preferably, the molar ratio of the (S)-4-amino-1-(pyridin-3-yl)butan-1-ol to the oxalyl chloride is 1: 1.

In the present application, the reaction temperature of Step S5 is 0 to 10° C.; and more preferably, the reaction temperature of Step S5 is 0° C.

In the present application, the reaction time of Step S5 is 20 to 40 min; and preferably, the reaction time of Step S5 is 30 min.

In the present application, at Step S5, extraction is required after the (S)-4-amino-1-(pyridin-3-yl)butan-1-ol reacts with the oxalyl chloride, and an extraction agent may be dichloromethane or ethyl acetate. After the extraction, an organic phase is taken and subjected to rotary evaporation for removing the solvent to obtain the (S)-4-amino-1-(pyridin-3-yl)chloro-butane.

In the present application, at Step S6, the (S)-4-amino-1-(pyridin-3-yl)chloro-butane prepared at Step S5 needs to be dissolved by adding tetrahydrofuran, and after the dissolution, a reaction with a base for cyclization is performed to form the S-demethylnicotine.

Preferably, at Step S6, the base is selected from one or more of sodium hydroxide, potassium hydroxide, lithium hydroxide, cesium hydroxide, barium hydroxide, and magnesium hydroxide; and more preferably, the base is sodium hydroxide.

In the present application, at Step S6, a molar ratio of the (S)-4-amino-1-(pyridin-3-yl)butyl-1-chloride to the sodium hydroxide is 1: (1.5-2.5); and preferably, the molar ratio of the (S)-4-amino-1-(pyridin-3-yl)butyl-1-chloride to the sodium hydroxide is 1: 2.

In the present application, at Step S6, the reaction temperature of the (S)-4-amino-1-(pyridin-3-yl)butyl-1-chloride and the base is 55 to 65° C., and the reaction time is 2 to 3 h; and preferably, the reaction temperature of the (S)-4-amino-1-(pyridin-3-yl)butyl-1 -chloride and the base is 60° C., and the reaction time is 2 h.

In the present application, a mixture containing S-demethylnicotine is obtained at Step S6.

In the present application, at Step S7, the amine methylation reagent is methyl iodide.

In the present application, at Step S7, a molar ratio of S-demethylnicotine in the mixture containing S-demethylnicotine to the methyl iodide is 1: (1.1-1.4); and preferably, the molar ratio of the S-demethylnicotine in the mixture containing S-demethylnicotine to the methyl iodide is 1: 1.2.

In the present application, at Step S7, the reaction temperature of the mixture containing S-demethylnicotine and the amine methylation reagent is 20 to 30° C., and the reaction time is 2 to 4 h; and preferably, the reaction temperature of the mixture containing S-demethylnicotine and the amine methylation reagent is 25° C., and the reaction time is 3 h.

In the present application, at Step S7, after the mixture containing S-demethylnicotine reacts with the amine methylation reagent, the pH needs to be adjusted to 6 by using an acid, extraction is performed, organic phases from four extractions are combined, dried over Na₂SO₄, and concentrated under reduced pressure to obtain crude S-nicotine, and the crude S-nicotine is purified by distillation to obtain the S-nicotine.

In summary, the present application has the following beneficial effects.

The present application provides a novel method for synthesizing S-nicotine by using cheap nicotinate and glutarate as starting materials, the raw materials are cheap, the cost is low, treatment processes in the reaction process are simple, the operation is easy, the reaction conditions are mild, the yield of S-nicotine is high, the ee value is high, and the reaction route is suitable for large-scale industrial production.

### DESCRIPTION OF THE EMBODIMENTS

The present application will be described in detail below in conjunction with embodiments.

The raw materials used in the present application can be obtained commercially, and if there is no special description, the raw materials not mentioned in the present application are purchased from Sinopharm Chemical Reagent Co., Ltd.

Embodiments 1 to 19 provide a preparation method for synthesizing S-nicotine from glutarate, which will be described below by taking Embodiment 1 as an example.

Embodiment 1 provides a preparation method for synthesizing S-nicotine from glutarate, wherein nicotinate is methyl nicotinate, the glutarate is diethyl glutarate, and a synthetic route is shown as Reaction Formula 2:

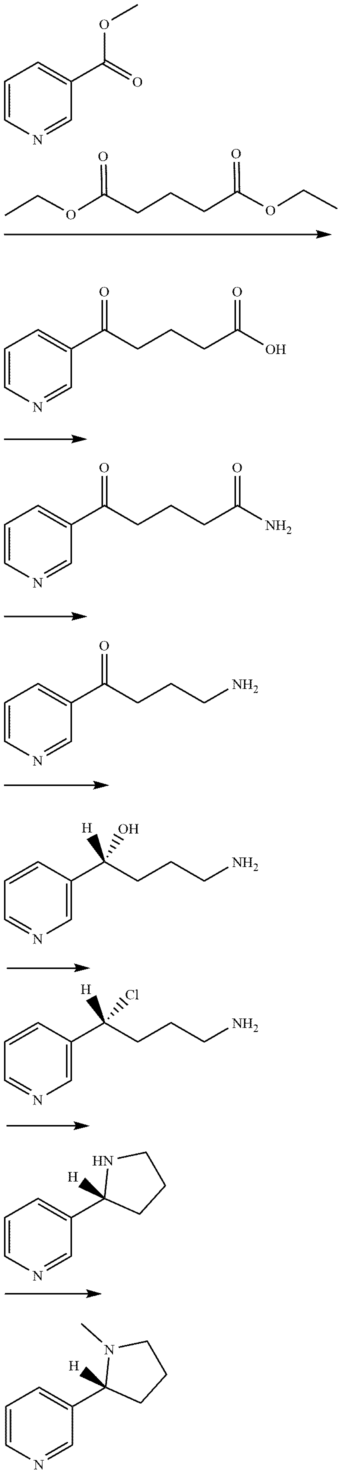

Specific preparation steps were as follows.
Step S1: 48 g (2 mol, 2 eq) of NaH and 282.3 g (1.5 mol, 1.5 eq) of diethyl glutarate were dissolved in 4 L of tetrahydrofuran at 0° C., a reaction was performed at 0° C. for 30 min, 137.1 g (1 mol) of methyl nicotinate was added, and a condensation reaction was performed at 25° C. and monitored by TCL until the end of the reaction to obtain a mixture containing 5-carbonyl-5-(pyridin-3-yl)pentanoic acid.
Step S2: a pH value of the mixture containing 5-carbonyl-5-(pyridin-3-yl)pentanoic acid prepared at Step S1 was adjusted to 4 by using 5 mol/L hydrochloric acid, and 204.36 g of industrial ammonium hydroxide (NH₃: 3 mol, 3 eq) with an NH₃ content of 25 wt% was added, and a reaction was performed at 65° C. for 2 h to obtain a mixture containing 5-oxo-5-(pyridin-3-yl)pentanamide.
Step S3: the mixture containing 5-oxo-5-(pyridin-3-yl)pentanamide obtained at Step S2 was quickly added into 111.7 g (1.5 mol, 1.5 eq) of sodium hypochlorite at 0° C., a reaction was performed at 0° C. for 1 h, the water bath was heated to 71° C., the reaction was continued at 71° C. for 1 h; after the reaction was stopped, the reaction solution was cooled to 25° C., the pH of the system was adjusted to 9 by using a saturated NaOH aqueous solution, after the solution turned black, ethyl acetate-water (a volume ratio of the ethyl acetate to the water was 1: 2) was added for three extractions, and an organic layer was taken, dried over absolute Na₂SO₄, filtered for removing the Na₂SO₄, subjected to rotary evaporation for removing the solvent, and dried under vacuum to obtain 4-amino-1-(pyridin-3-yl)butanone.
Step S4: the 4-amino-1-(pyridin-3-yl)butanone obtained at Step S3 was dissolved in 5 L of tetrahydrofuran at 0° C., 481.1 g (1.5 mol, 1.5 eq) of (+)-B-diisopinocampheyl chloroborane was added, and a reaction was performed at 0° C. for 2 h to obtain a mixture containing (S)-4-amino-1-(pyridin-3-yl)butan-1-ol;
Step S5: 126.9 g (1 mol, 1 eq) of oxalyl chloride was added into the mixture containing (S)-4-amino-1-(pyridin-3-yl)butan-1-ol obtained at Step S4 at 0° C., a reaction was performed at 0° C. for 30 min, after the reaction, extraction was performed by using dichloromethane, and an organic phase was taken and subjected to rotary evaporation for removing the solvent to obtain (S)-4-amino-1-(pyridin-3-yl)chloro-butane.
Step S6: 2 L of tetrahydrofuran was added into the (S)-4-amino-1-(pyridin-3-yl) chloro-butane obtained at Step S5, after the dissolution, 80 g (2 mol, 2 eq) of NaOH was added, and after the dissolution by stirring, a reaction was performed at 60° C. for 2 h to obtain a mixture containing S-demethylnicotine.
Step S7: 170.3 g (1.2 mol, 1.2 eq) of methyl iodide was added into the mixture containing S-demethylnicotine prepared at Step S6, a reaction was performed at 25° C. for 3 h, the pH of the system was adjusted to 6 by using 5 mol/L hydrochloric acid, extraction was performed by using dichloromethane, an organic phase was taken, added with Na₂SO₄ for drying, and concentrated under reduced pressure for removing the solvent to obtain crude S-nicotine; and the crude S-nicotine was further purified once by atmospheric distillation to obtain S-nicotine with a yield of 53%, an ee value of 98%, and a purity of 95%.

It is worthwhile to note that each mass and specific molar weight in the embodiments of the present application can be selected according to the size of an industrially produced vessel as long as the equivalence ratio of each reaction raw material is consistent.

A difference between Embodiments 2 to 3 and Embodiment 1 is that: in the reaction of Step S1, the kind of the base catalyst was adjusted as specifically shown in Table 1.

Table 1

Effect of selection of base catalyst on the reaction of Step S 1

| Serial number | Selection of base catalyst | Yield of S-nicotine (%) |
|---|---|---|
| Embodiment 1 | NaH | 53 |
| Embodiment 2 | Sodium tert-butoxide | 50 |
| Embodiment 3 | Potassium tert-butoxide | 50 |

A difference between Embodiments 4 to 5 and Embodiment 1 is that: in the reaction of Step S1, the usage amounts of the diethyl glutarate and the NaH were adjusted as specifically shown in Table 2.

Table 2

Effect of usage amounts of diethyl glutarate and NaH on the reaction of Step S1

| Serial number | Equivalent quantity (eq) of diethyl glutarate (eq) | Amount of substance of NaH (mol) | Yield of S-nicotine (%) |
|---|---|---|---|
| Embodiment 1 | 1.5 | 2 | 53 |
| Embodiment 4 | 1.5 | 1.2 | 45 |
| Embodiment 5 | 1 | 1.2 | 48 |

A difference between Embodiments 6 to 7 and Embodiment 1 is that: in the reaction of Step S2, the usage amount of the ammonium hydroxide was adjusted as specifically shown in Table 3.

Table 3

Effect of usage amount of ammonium hydroxide on the reaction of Step S2

| Serial number | Equivalent quantity (eq) of ammonium hydroxide | Yield of S-nicotine (%) |
|---|---|---|
| Embodiment 1 | 3 | 53 |
| Embodiment 6 | 2 | 45 |
| Embodiment 7 | 4 | 48 |

A difference between Embodiments 8 to 9 and Embodiment 1 is that: in the reaction of Step S4, the usage amount of the (+)-B-diisopinocampheyl chloroborane was adjusted as specifically shown in Table 4.

Table 4

Effect of usage amount of (+)-B-diisopinocampheyl chloroborane on the reaction of Step S4

| Serial number | Equivalent quantity (eq) of (+)-B-diisopinocampheyl chloroborane | Yield of S-nicotine (%) |
|---|---|---|
| Embodiment 1 | 1.5 | 53 |
| Embodiment 8 | 1 | 49 |
| Embodiment 9 | 2 | 51 |

A difference between Embodiments 10 to 12 and Embodiment 1 is that: in the reaction of Step S4, the reaction temperature was adjusted as specifically shown in Table 5.

Table 5

Effect of reaction temperature on the reaction of Step S4

| Serial number | Reaction temperature (°C) | Yield of S-nicotine (%) |
|---|---|---|
| Embodiment 1 | 0 | 53 |
| Embodiment 10 | -10 | 45 |
| Embodiment 11 | 10 | 50 |
| Embodiment 12 | 5 | 49 |

A difference between Embodiments 13 to 15 and Embodiment 1 is that: in the reaction of Step S4, the kind of the organic solvent was adjusted as specifically shown in Table 6.

Table 6

Effect of selection of organic solvent on the reaction of Step S4

| Serial number | Selection of organic solvent | Yield of S-nicotine (%) |
|---|---|---|
| Embodiment 1 | Tetrahydrofuran | 53 |
| Embodiment 13 | 1,4-dioxane | 25 |
| Embodiment 14 | Methyl tertiary butyl ether | 0 |
| Embodiment 15 | Absolute ether | 0 |

A difference between Embodiments 16 to 17 and Embodiment 1 is that: in the reaction of Step S5, the usage amount of the oxalyl chloride was adjusted as specifically shown in Table 7.

Table 7

Effect of usage amount of oxalyl chloride on the reaction of Step S5

| Serial number | Equivalent quantity (eq) of oxalyl chloride | Yield of S-nicotine (%) |
|---|---|---|
| Embodiment 1 | 1 | 53 |
| Embodiment 16 | 1.5 | 49 |
| Embodiment 17 | 0.5 | 41 |

A difference between Embodiment 18 and Embodiment 1 is that: at Step S1, the methyl nicotinate was replaced with equimolar ethyl nicotinate, and prepared S-nicotine had a yield of 52%, an ee value of 98%, and a purity of 95%.

A difference between Embodiment 19 and Embodiment 1 is that: at Step S1, the diethyl glutarate was replaced with equimolar dimethyl glutarate, and prepared S-nicotine had a yield of 54%, an ee value of 98%, and a purity of 95%.

The specific embodiments are merely an explanation of the present application and are not intended to limit the present application. After reading the present description, those skilled in the art can make modifications to the present embodiments as required without any inventive contribution, and these modifications shall fall within the scope of protection of the present application.

What is claimed is:

1. A preparation method for synthesizing S-nicotine from glutarate, comprising the following steps:
step S1: performing a condensation reaction on nicotinate and glutarate in the presence of a base catalyst to obtain 5-carbonyl-5-(pyridin-3-yl)pentanoic acid;
step S2: reacting the 5-carbonyl-5-(pyridin-3-yl)pentanoic acid with an amination reagent to obtain 5-oxo-5-(pyridin-3-yl)pentanamide;
step S3: performing a Hofmann degradation reaction on the 5-oxo-5-(pyridin-3-yl)pentanamide in the presence of hypochlorite to obtain 4-amino-1-(pyridin-3-yl)butanone;
step S4: adding the 4-amino-1-(pyridin-3-yl)butanone and (+)-B-diisopinocampheyl chloroborane into an organic solvent, and reacting at -30 to 10° C. to obtain (S)-4-amino-1-(pyridin-3 -yl)butan-1-ol;
step S5: reacting the (S)-4-amino-1-(pyridin-3-yl)butan-1-ol with a chlorination reagent to obtain (S)-4-amino-1-(pyridin-3-yl)chloro-butane;
step S6: performing a cyclization reaction on (S)-4-amino-1-(pyridin-3-yl)butyl-1-chloride in the presence of a base to obtain S-demethylnicotine; and
step S7. reacting the S-demethylnicotine with an amine methylation reagent to obtain the S-nicotine.

2. The preparation method for synthesizing S-nicotine from glutarate according to claim 1, wherein, at the step S1, a molar ratio of the nicotinate to the glutarate to the base catalyst is 1: (1-1.5): (1.2-2).

3. The preparation method for synthesizing S-nicotine from glutarate according to claim 1, wherein, at the step S2, the amination reagent is one or more selected from a group consisting of ammonium hydroxide, formamide, and acetamide.

4. The preparation method for synthesizing S-nicotine from glutarate according to claim 3, wherein, at the step S2, a molar ratio of the 5-carbonyl-5-(pyridin-3-yl)pentanoic acid to the ammonium hydroxide is 1: (2-4).

5. The preparation method for synthesizing S-nicotine from glutarate according to claim 1, wherein, at the step S3, a molar ratio of the 5-oxo-5-(pyridin-3-yl)pentanamide to the hypochlorite is 1: (1-2).

6. The preparation method for synthesizing S-nicotine from glutarate according to claim 1, wherein, at the step S4, a molar ratio of the 4-amino-1-(pyridin-3-yl)butanone to the (+)-B-diisopino- campheyl chloroborane is 1: (1.2-2).

7. The preparation method for synthesizing S-nicotine from glutarate according to claim 6, wherein, at the step S4, the organic solvent is tetrahydrofuran.

8. The preparation method for synthesizing S-nicotine from glutarate according to claim 6, wherein, a reaction temperature of the step S4 is 0° C.

9. The preparation method for synthesizing S-nicotine from glutarate according to claim 1, wherein, at the step S5, the chlorination reagent is one or more selected from a group consisting of oxaloyl chloride, thionyl chloride, and trichlorophosphorus.

10. The preparation method for synthesizing S-nicotine from glutarate according to claim 9, wherein, at the step S5, a molar ratio of the (S)-4-amino-1-(pyridin-3-yl)butan-1-ol to the oxaloyl chloride is 1: (1-1.5).

11. The preparation method for synthesizing S-nicotine from glutarate according to claim 7, wherein, a reaction temperature of the step S4 is 0° C.
